(19) United States
(12) Patent Application Publication
(10) Pub. No.: US 2008/0140024 A1
(43) Pub. Date: Jun. 12, 2008

(54) Drug delivery device

(76) Inventor: Yoseph Yaacobi, Fort Worth, TX (US)

Correspondence Address: ALCON, IP LEGAL, TB4-8, 6201 SOUTH FREEWAY, FORT WORTH, TX, 76134, US

(21) Appl. No.: 11/876,268

(22) Filed: Oct. 22, 2007

Related U.S. Application Data
(60) Provisional application No. 60/869,141, filed on Dec. 8, 2006.

(51) Int. Cl. A61M 31/00

(52) U.S. Cl. 604/285

(57) ABSTRACT

Drug delivery devices, and methods of delivering pharmaceutically active agents to a target tissue within a body using such devices, are disclosed. One drug delivery device includes a body having an internal surface for disposing proximate a target tissue and a well having an opening to the internal surface. An inner core is disposed within the well. The inner core comprises a pharmaceutically active agent and a tunnel leading into the interior of the inner core.

This application claims the priority of U.S. Provisional Application Ser. No. 60/869,141 filed on Dec. 8, 2006.

### FIELD OF THE INVENTION

The present invention generally pertains to biocompatible implants for localized delivery of pharmaceutically or biologically active agents to body tissue. More particularly, but not by way of limitation, the present invention pertains to biocompatible implants for localized delivery of pharmaceutically active agents to the posterior segment of the eye.

2. Description of the Related Art

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

Age related macular degeneration (ARMD) is the leading cause of blindness in the elderly. ARMD attacks the center of vision and blurs it, making reading, driving, and other detailed tasks difficult or impossible. About 200,000 new cases of ARMD occur each year in the United States alone. Current estimates reveal that approximately forty percent of the population over age 75, and approximately twenty percent of the population over age 60, suffer from some degree of macular degeneration. “Wet” ARMD is the type of ARMD that most often causes blindness. In wet ARMD, newly formed choroidal blood vessels (choroidal neovascularization (CNV)) leak fluid and cause progressive damage to the retina.

In the particular case of CNV in ARMD, three main methods of treatment are currently being developed, (a) photocoagulation, (b) the use of angiogenesis inhibitors, and (c) photodynamic therapy. Photocoagulation is the most common treatment modality for CNV. However, photocoagulation can be harmful to the retina and is impractical when the CNV is near the fovea. Furthermore, over time, photocoagulation often results in recurrent CNV. Oral or parenteral (non-ocular) administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required. Periocular injections of these compounds often result in the drug being quickly washed out and depleted from the eye, via periocular vasculature and soft tissue, into the general circulation. Repetitive intraocular injections may result in severe, often blinding, complications such as retinal detachment and endophthalmitis. Photodynamic therapy is a new technology for which the long-term efficacy is still largely unknown.

In order to prevent complications related to the above-described treatments and to provide better ocular treatment, researchers have suggested various implants aimed at localizing delivery of anti-angiogenic compounds to the eye. U.S. Pat. No. 5,824,072 to Wong discloses a non-biodegradable polymeric implant with a pharmaceutically active agent disposed therein. The pharmaceutically active agent diffuses through the polymer body of the implant into the target tissue. The pharmaceutically active agent may include drugs for the treatment of macular degeneration and diabetic retinopathy. The implant is placed substantially within the tear fluid upon the outer surface of the eye over an avascular region, and may be anchored in the conjunctiva or sclera; episclerally or intrasclerally over an avascular region; substantially within the suprachoroidial space over an avascular region such as the pars plana or a surgically induced avascular region; or in direct communication with the vitreous.

U.S. Pat. No. 5,476,511 to Gwon et al. discloses a polymer implant for placement under the conjunctiva of the eye. The implant may be used to deliver neovascular inhibitors for the treatment of ARMD and drugs for the treatment of retinopathies, and retinitis. The pharmaceutically active agent diffuses through the polymer body of the implant.

U.S. Pat. No. 5,773,019 to Ashton et al. discloses a non-bioerodable polymer implant for delivery of certain drugs including angiostatic steroids and drugs such as cyclosporine for the treatment of uveitis. Once again, the pharmaceutically active agent diffuses through the polymer body of the implant.

All of the above-described implants require careful design and manufacture to permit controlled diffusion of the pharmaceutically active agent through a polymer body (i.e., matrix devices) or polymer membrane (i.e., reservoir devices) to the desired site of therapy. Drug release from these devices depends on the porosity and diffusion characteristics of the matrix or membrane, respectively. These parameters must be tailored for each drug moiety to be used with these devices. Consequently, these requirements generally increase the complexity and cost of such implants.

U.S. Pat. No. 5,824,073 to Peyman discloses an indentor for positioning in the eye. The indentor has a raised portion that is used to indent or apply pressure to the sclera over the macular area of the eye. This patent discloses that such pressure decreases choroidal congestion and blood flow through the subretinal neovascular membrane, which, in turn, decreases bleeding and subretinal fluid accumulation.

Therefore, a need exists in the biocompatible implant field for a surgically implantable drug delivery device capable of safe, effective, rate-controlled, localized delivery of a wide variety of pharmaceutically active agents. The surgical procedure for implanting such a device should be safe, simple, quick, and capable of being performed in an outpatient setting. Ideally, such a device should be easy and economical to manufacture. Furthermore, because of its versatility and capability to deliver a wide variety of pharmaceutically active agents, such an implant should be capable of use in clinical studies to deliver various agents that create a specific physical condition in a patient. In the particular case of ophthalmic drug delivery, such an implantable drug delivery device is especially needed for localized delivery of pharmaceutically active agents to the posterior segment of the eye to combat ARMD, CNV, retinopathies, retinitis, uveitis, macular edema, glaucoma, and neuropathies.

### SUMMARY OF THE INVENTION

In one aspect, the present invention is a drug delivery device including a body having an internal surface for disposing proximate a target tissue and a well having an opening to the internal surface. The device includes an inner core disposed in the well. The inner core includes a pharmaceutically active agent, a first surface proximate the internal surface, and a tunnel fluidly coupling the first surface to an interior of the inner core. The tunnel allows a fluid from the target tissue to contact the interior of the inner core.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a schematic side sectional view of a drug delivery device having a fenestrated inner core according to a preferred embodiment of the present invention;

FIGS. 2a-2c are schematic side sectional views of alternate embodiments of the drug delivery device of FIG. 1; and

FIGS. 3-4 are schematic side sectional views of alternate embodiments of the drug delivery device of FIG. 2c.

### DETAILED DESCRIPTION OF THE PREFERRED EMBODIMENTS

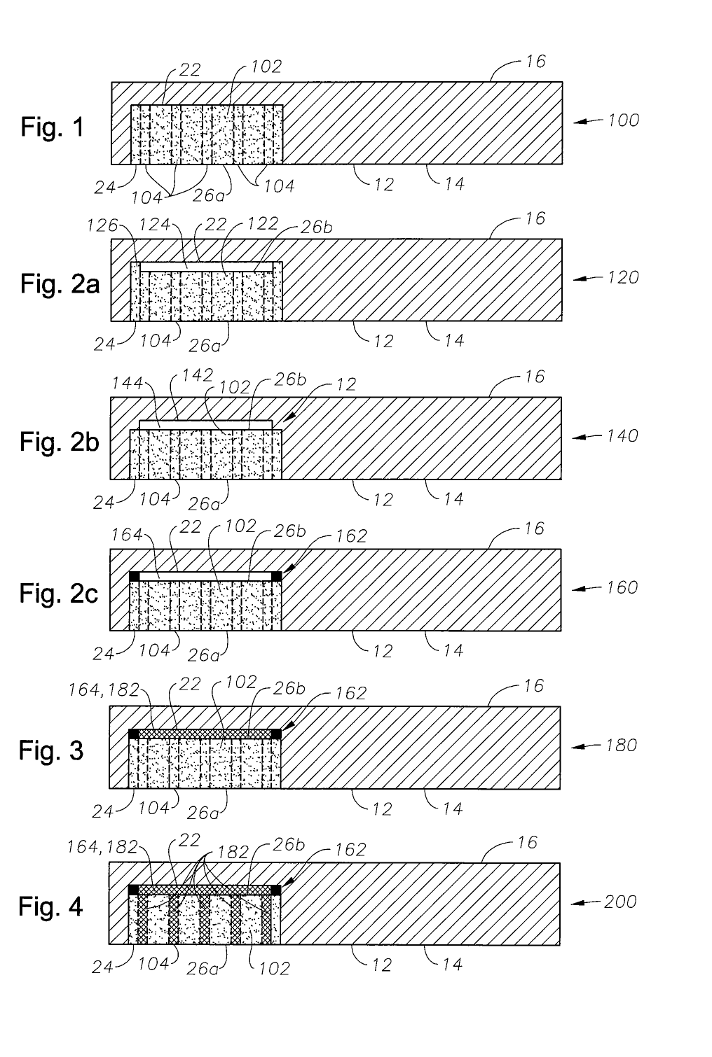

The preferred embodiments of the present invention and their advantages are best understood by referring to FIGS. 1-4 of the drawings, like numerals being used for like and corresponding parts of the various drawings.

FIG. 1 schematically illustrates a drug delivery device 100 according to a preferred embodiment of the present invention. Device 100 may be used in any case where localized delivery of a pharmaceutically active agent, or a biologically active agent, to body tissue is required. By way of example, device 100 may be used to treat a medical disorder of the eye, ear, nose, throat, skin, subcutaneous tissue, or bone. Device 100 may be used in humans or animals. Drug delivery device 100 is substantially similar in construction and operation to drug delivery device 10 described in U.S. Pat. No. 6,413,540, with the exception of inner core 102 of device 100. U.S. Pat. No. 6,413,540 (the “'540 Patent”) is commonly owned with the subject application and is incorporated herein by reference. Unless otherwise specified, terms and definitions used herein have the same meaning as provided in the '540 Patent. Although drug delivery device 100 is described herein in connection with drug delivery device 10 of the '540 Patent, the present invention is also applicable to drug delivery devices 10a and 50 described in '540 Patent as well as other drug delivery devices.

Drug delivery device 100 has a body 12 made from a biocompatible, nonbioerodible material and an inner core 102 comprising a pharmaceutically active agent, or a biologically active agent, suitable for localized delivery to a target tissue. A preferred material for body 12 is silicone. Body 12 has an internal surface 14 and an external surface 16. Body 12 includes a well or cavity 22 having an opening 24 to internal surface 14. Inner core 102 is disposed in well 22. Inner core 102 may comprise any of the forms of inner core 26 of drug delivery device 10 or inner core 106 of drug delivery device 50 of the '540 Patent. Inner core 102 is preferably a tablet comprising a pharmaceutically active agent. Inner core 102 has a surface 26a proximate opening 24 of internal surface 14.

Inner core 102 is preferably formed with one or more tunnels or channels 104 that fluidly couple surface 26a and the interior of inner core 102. Tunnels allow fluid from the target tissue to contact portions of the interior of inner core 102 in addition to surface 26a. Tunnels 104 may be formed via any mechanical, chemical, electrical, optical, or thermal means. Tunnels 104 are preferably formed by using a laser.

Device 100 is preferably surgically placed proximate a target tissue, as is more fully described in the '540 Patent. The physical shape of body 12, including the geometry of internal surface 14, well 22, opening 24, facilitate the unidirectional delivery of a pharmaceutically effective amount of the pharmaceutically active agent from inner core 102 to the target tissue. The absence of a polymer layer or membrane between inner core 102 and the underlying tissue greatly enhances and simplifies the delivery of an active agent to the target tissue. In particular, it has been discovered that tunnels 104 increase the bioavailability of the pharmaceutically active agent in the target tissue without the necessity of increasing the size of inner core 102, increasing the concentration of the pharmaceutically active agent within inner core 102, or adding excipients to inner core 102 that reduce the amount of the pharmaceutically active agent and thus the duration of agent release of device 100.

Device 100 can be used to deliver a pharmaceutically effective amount of a pharmaceutically active agent to target tissue for many years, depending on the particular physicochemical properties of the agent employed. Important physicochemical properties include hydrophobicity, solubility, dissolution rate, diffusion coefficient, and tissue affinity. It addition, it has been discovered that tunnels 104 result in additional lateral diffusion of the pharmaceutically active agent to areas within the target tissue not directly below inner core 102.

FIGS. 2a-2c illustrate three alternative embodiments of drug delivery device 100 of FIG. 1. Drug delivery device 120 of FIG. 2a has an inner core 122 that is substantially similar in construction and operation to inner core 102 of device 100, with the exception that inner core 122 is formed with a cavity 124 on its external end 126. Cavity 124 creates an additional surface 26b for fluid from the target tissue to contact. Drug delivery device 140 of FIG. 2b is substantially similar in construction and operation to drug delivery device 100, with the exception that device 140 includes a body 12 having a well 142 with a cavity 144. Cavity 144 creates an additional surface 26b for fluid from the target tissue to contact. Drug delivery device 160 of FIG. 2c is substantially similar in construction and operation to drug delivery device 100, with the exception that device 160 includes a spacing member 162 that creates a cavity 164 within well 22. Spacing member 162 preferably has a ring-shaped geometry. Cavity 164 creates an additional surface 26b for fluid from the target tissue to contact.

FIGS. 3-4 illustrate two alternative embodiments of drug delivery device 160 of FIG. 2c. Drug delivery device 180 of FIG. 3 is identical in construction and operation to device 160, with the exception that cavity 164 contains a fluid attracting material 182. Drug delivery device 200 of FIG. 4 is identical in construction and operation to device 160, with the exception that cavity 164 and tunnels 104 contain fluid attracting material 182. Fluid attracting material 182 may be any material that attracts fluid from the target tissue. Fluid attracting material 182 may also contain an additional pharmaceutically active agent and/or biologically active agent other than the agent within inner core 102. Fluid attracting material 182 may also contain a penetration enhancer or other excipient aimed at improving the bioavailability of a pharmaceutically active agent or biologically active agent within the target tissue. Although not shown in the FIGURES, fluid attracting material 182 may also be incorporated into devices 100, 120, and 140.

It is believed that devices 120, 140, 160, 180, and 200 further increase the bioavailability of the pharmaceutically active agent or biologically active agent of inner core 122 in the target tissue.

From the above, it may be appreciated that the present invention provides improved devices and methods for safe, effective, rate-controlled, localized delivery of a variety of pharmaceutically active agents to any body tissue. The surgical procedure for implanting such devices is safe, simple, quick, and capable of being performed in an outpatient setting. Such devices are easy and economical to manufacture. Furthermore, because of their capability to deliver a wide variety of pharmaceutically active agents, such devices are useful in clinical studies to deliver various agents that create a specific physical condition in a patient or animal subject. In the particular field of ophthalmic drug delivery, such devices are especially useful for localized delivery of pharmaceutically active agents to the posterior segment of the eye to combat ARMD, CNV, retinopathies, retinitis, uveitis, macular edema, and glaucoma.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims

What is claimed is:

1. A drug delivery device, comprising:
a body having an internal surface for disposing proximate a target tissue and a well having an opening to said internal surface; and
an inner core disposed in said well, comprising:
a pharmaceutically active agent;
a first surface proximate said internal surface; and
a tunnel fluidly coupling said first surface to an interior of said inner core;
whereby said tunnel allows a fluid from said target tissue to contact said interior of said inner core.

2. The drug delivery device of claim 1 wherein said inner core has a second surface opposite said first surface and a cavity disposed on said second surface, said cavity allowing said fluid from said target tissue to contact said second surface.

3. The drug delivery device of claim 1 wherein:
said inner core has an second surface opposite said first surface;
said well includes a cavity formed proximate said second surface; and
said cavity allows said fluid from said target tissue to contact said second surface.

4. The drug delivery device of claim 1 wherein said inner core has a second surface opposite said first surface, and further comprising a spacing member disposed in said well proximate said second surface, whereby said spacing member forms a cavity that allows fluid from said target tissue to contact said second surface.

5. The drug delivery device as in any of claims 2-4 further comprising a fluid attracting material disposed in said cavity.

6. The drug delivery device of claim 5 further comprising said fluid attracting material disposed in said tunnel.

7. The drug delivery device of claim 1 further comprising a plurality of said tunnels.

8. The drug delivery device of claim 1 wherein said inner core is a tablet.
